(19) United States
(12) Patent Application Publication
(10) Pub. No.: US 2024/0008857 A1
(43) Pub. Date: Jan. 11, 2024

(54) DEVICE FOR THE IN-SITU ANALYSIS OF EXCREMENT, SAMPLE-CARRIER CARTRIDGE FOR SUCH A DEVICE AND ALSO TOILET UNIT AND TOILET SEAT

(71) Applicant: MEDIPEE GMBH, Moers (DE)

(72) Inventors: Thomas PROKOPP, Bonn (DE); Paul BANDI, Kamp-Lintfort (DE); Frank WILLEMS, Moers (DE); Jan HAAN, Moers (DE); Marcel MORCINCZYK, Rheinberg (DE); Niklas KÜRTEN, Langenfeld (DE)

(21) Appl. No.: 18/039,512

(22) PCT Filed: Nov. 26, 2021
(86) PCT No.: PCT/EP2021/083190

§ 371 Date: May 31, 2023

(30) Foreign Application Priority Data
Dec. 7, 2020 (DE) 10 2020 132 506.1

(51) Int. Cl. A61B 10/00; G06T 7/00; E03D 11/13; G06T 7/60

(52) U.S. Cl. CPC A61B 10/0038; A61B 10/007; G06T 7/0012; E03D 11/13; G06T 7/60

(57) ABSTRACT

The invention relates to a device for on-site analysis of excrements by means of a sensor device with which an excrement sample can be detected at a measuring location and at least partially analyzed, the sensor device having a bendable sensor arm, in particular for a sample carrier, which can be displaced in the longitudinal direction by an actuator between an extended measuring position, bridging a distance between the holder and the measuring location for collecting the excrement sample, and a retracted mounting position, at least partially bent, on the holder.

The invention further relates to a self-sealing cartridge for the provision of sample carriers for a device for on-site analysis of excrements, having a cartridge housing receiving the sample carriers which is formed by a first housing half and a second housing half, these two being displaceable in relation to one another between a closed position and a dispensing position.

The invention further relates to a toilet device for analyzing excrements, comprising the collecting device according to the invention for collecting excrements and further comprising a toilet seat for a toilet device, a device for on-site analysis of the excrements collected in the toilet bowl being accommodated in the toilet seat.

The invention relates to a device for on-site analysis of excrement with a sensor device by means of which an excrement sample can be detected at a measuring location, the sensor device having a bendable, preferably elastically bendable, sensor arm which can be mounted on a holder at least partially bent and can be displaced between a measuring position and a retracted position.

The invention likewise relates to a cartridge for providing sample carriers for an apparatus for on-site analysis of excrement, the cartridge having a cartridge housing accommodating the sample carriers.

The invention further relates to a toilet device for analyzing excrement, the toilet device comprising a collection device for collecting the excrement.

The invention also relates to a toilet seat for a toilet device comprising a toilet bowl for collecting excrements.

It is known that by means of analyses of urine and/or stool samples, at least initial findings concerning a state of health of an organism, in particular a human being, can be determined. However, in order to obtain sufficiently meaningful analysis information, it is usually necessary to safely transfer urine and/or stool samples taken at a toilet to a transport vessel in order to send this vessel with the sample contained therein to an analysis laboratory. This is not only inconvenient, but can also take longer before the corresponding analysis results are available. However, it is particularly disadvantageous here that close monitoring in the sense of continuous preventive examinations is generally not carried out because of the considerable effort involved. In this respect, it is desirable to create a solution that facilitates the analysis of excrements.

The applicant's own patent application DE 10 2017 011 883 A1 proposes, for example, a device for on-site analysis of excrement of the generic type described at the beginning, which can be attached to a toilet bowl of a toilet and can analyze the excrement at least partially already in/at the toilet bowl. A sensor arm is formed here from a strip of material that can be rolled up or unrolled, which can be unrolled from a roll to extend the sensor arm and rolled up again onto the roll to retract it. The described analysis device offers a considerable time advantage compared to an analysis of samples to be sent first to an analysis laboratory and is much easier to employ for a user, because the excrement samples do not have to be brought into a transport vessel for analysis. However, it has been shown that, particularly during continuous operation of the device, disturbances can occur during unrolling or rolling of the sensor arm from or onto the roll, which can lead to an impairment of the functional retraction and extension of the sensor arm.

Against this background, the invention is based on the task of providing an improved, compactly designed device for analyzing excrement, especially human excrement, which operates reliably. Disturbances during the retraction and extension of the sensor arm should be reliably prevented, so that the sensor arm can always be brought precisely to the desired measuring location, which at the same time increases the measuring accuracy and enables a reliable analysis. Furthermore, the invention is based on the task of providing a cartridge for the provision of sample carriers for a device for on-site analysis of excrements, which is likewise directed at reliable operation and which enables reliable and accurate analysis at all times. In this context, it is also intended to provide a toilet device for analyzing excrement, in particular human excrement, and a toilet seat for a toilet device with a toilet bowl for collecting excrement, in particular human excrement, to enable reliable and trouble-free analysis operation and to be compact in design.

This task is solved by a device for on-site analysis of excrement having the features of claim 1, by a cartridge for providing sample carriers for a device for on-site analysis of excrement having the features of claim 21, by a toilet device for analysis of excrement having the features of claim 24, and by a toilet seat for a toilet device having a toilet bowl for collecting excrement having the features of claim 30. Further, particularly advantageous embodiments of the invention are disclosed by the respective dependent claims.

It should be noted that the features listed individually in the claims may be combined with one another in any technically useful manner (even across category boundaries, for example between method and device) and reveal further embodiments of the invention. The specification additionally characterizes and specifies the invention, particularly in connection with the figures.

It should also be noted that the expression “and/or” used herein, positioned between two features and linking them, is always to be interpreted in such a way that in a first embodiment of the subject matter of the invention only the first feature can be present, in a second embodiment only the second feature can be present, and in a third embodiment both the first and the second feature can be present.

According to the invention, a device for on-site analysis of excrement, in particular human excrement, has a sensor device by means of which an excrement sample can be collected at a measurement location and can be at least partially analyzed. The sensor device has a bendable, preferably elastically bendable, on a holder at least partially bent-mounted sensor arm, in particular for a sample carrier, which can be displaced in the longitudinal direction by an actuator between an extended measuring position, bridging a distance between the holder and the measuring location for collecting the excrement sample, and a retracted mounting position, at least partially bent, on the holder. The invention further provides for the sensor arm to be maintained at a distance, via at least one spacing means, from such portions of the holder (also referred to herein as critical portions) relative to which the sensor arm moves in the direction of its longitudinal displacement.

In other words, the spacing means effectively prevents frictional contact during the longitudinal displacement of the sensor arm between the measuring position and the mounting position, which would become effective in the event of direct contact of the sensor arm with said static holder portions, which are in particular fixed in relation to the sensor arm, between these and the sensor arm. This reduces the driving force to be applied by the actuator to displace the sensor arm. The sensor arm can be moved more easily into the measuring position without the frictional contact to said holding parts, whereby, for example, a sample carrier held by the sensor arm can always be moved precisely and reliably to the measuring location for collecting the excrements, which in turn reduces the probability that the analysis results will be falsified. Accordingly, the analysis results that can be provided by the sensor device improve in terms of their accuracy and reliability, among other things.

Disturbances in the operation of the on-site analysis device according to the invention, in particular during the retraction and extension of the sensor arm, are completely avoided or at least significantly reduced. For example, if the sensor arm is pushed rather than pulled by the actuator to be extended to the measurement or mounting position, the elimination of direct frictional contact between the sensor arm and the corresponding parts of the holder achieves that the sensor arm cannot get stuck and stalled on the holder or parts thereof. Stalling of the sensor arm while the actuator is advancing can cause the sensor arm to become entangled if, for example, several sections of the sensor arm that are bent on the holder cross each other or if the sensor arm is suddenly bent in an uncontrolled manner in one arm section due to the advancing force that continues to act and, as a result, becomes jammed with the holder or with other sensor arm sections.

In total, the spacing according to the invention ensures substantially frictionless longitudinal displacement of the sensor arm relative to the holder or critical portions thereof that could cause possible frictional contact with the longitudinally displacing sensor arm. In particular, the spacing means prevents direct, immediate contact between the sensor arm and such portions of the holder.

For the purposes of the invention, the term “sensor device” covers all devices which are suitable for sensing and at least partially analyzing a sample of excrement. For this purpose, the sensor device may comprise at least one sensor, preferably an entire sensor unit with a plurality of identical or different sensors. Here, the sensor or sensors can, for example, sensorially detect a sample carrier for the analysis that has previously interacted with the excrement sample. It is understood that different sensors can also be provided for this purpose depending on the analysis variable to be evaluated. For example, the sensor unit can have a color sensor for detecting a color change of the sample carrier, whereby an initial analysis in the sense of the invention is already performed. Alternative or additional sensors may comprise, for example, touch sensors, ultrasonic sensors, temperature sensors, pressure sensors, and the like, but are not necessarily limited thereto.

Additionally or alternatively, data may be transmitted from the sensor unit and/or from the sensor device to an analysis unit external to the device, for example to a data processing and/or analysis application executed on a smartphone or the like. The transmission may be wireless or wired, with the transmission between the on-site analysis device and the external data processing and/or analysis application or the like preferably being wireless. Advantageously, the sensor device and/or the additional, device-external analysis unit for the analysis has a suitable electronic computing and control unit, for example a microprocessor, microcontroller or the like, as well as a corresponding electronic memory unit, for example ROM, RAM, flash memory, etc., by means of which, in the simplest embodiment, for example a color change on the sample carrier can be analyzed. It is conceivable here that at least some of the sensors can be switched off or on depending on the excrement to be analyzed; in this manner the device for on-site analysis can be operated more efficiently in terms of energy overall.

The term “sample carrier” is to be understood as a piece of material suitable for detecting excrement, such as an indicator strip or the like.

For purposes of the invention, the term “excrement” comprises bodily excretions, such as urine and faeces.

The term “on-site analysis” describes in the sense of the invention that an analysis of excrement can be carried out at least partially or preferably entirely at the place of excretion, such as for example at a toilet, urinal or the like. In other words, this means that the excrement to be analyzed, of whatever kind, does not have to be sent to a distant laboratory for analysis. It is understood, however, that data or information obtained on site can also be transmitted to external facilities, such as a data carrier, etc., for further evaluation or storage. By means of on-site analysis it is possible, for example, to determine a wide variety of urine or micturition parameters.

To analyze a desired excrement sample, the movable sensor arm is extended from the holder to such an extent that it can come into contact with the respective excrement at the measurement location, for example with a sample carrier held by it, i.e. it can detect the excrement.

Subsequently, the sample carrier wetted with the excrement is examined by means of the sensor or sensors of the sensor device. The analysis of the sample by means of the at least one sensor is preferably performed directly at the measuring location, i.e., while the sensor arm is in its (fully) extended measuring position. In this case, however, the actual sensor for analyzing the sample may be mounted remotely from the measurement location, for example in local proximity to the holder of the sensor arm. For example, the sensor can be a camera that analyzes the sample carrier wetted with excrement from a distance, for example registers a possible color change on an indicator strip (sample carrier). In any case, the sensor arm does not have to be retracted toward its mounting position first to perform the analysis. This reliably prevents contamination of at least part of the sensor device, e.g. the analysis sensor(s), the holder, etc., with excrement.

Particularly preferably, the sensor arm is elastically bendable so that it essentially automatically moves from the rest position bent on the holder into an elongated state of its measuring position for reaching the measuring location, which, among other things, increases the positioning accuracy of the sensor arm at the measuring location and thus also the positioning accuracy of a sample carrier held, for example, by the sensor arm at the measuring location for detecting the excrement. The precise guidance of the sensor arm to the measuring location achieved in this way enables the use of an actuator of simple and compact design. For example, only a single actuator generating a relatively low drive force can be provided, which advances the sensor arm to one position (e.g., measuring position) and retracts it to the other position (e.g., mounting position).

An advantageous embodiment of the invention provides for the sensor arm to be wire-shaped.

For example, the sensor arm may be a wire made of a metal material (e.g., stainless steel, copper, brass, aluminum and the like, and alloys thereof) or a wire made of a plastic material (e.g., nylon wire, PE wire, PVC wire, etc.). In any case, the wire-shaped sensor arm has a tensile and compressive stiffness effective in the longitudinal direction, whereas it can be bent transversely to its longitudinal direction, preferably elastically. The sensor arm may have different cross-sections, for example round (e.g., circular, elliptical, lenticular, etc.) or angular (polygonal with three, four, five or more corners). The sensor arm may also be ribbon-shaped, meaning that the cross-section of the sensor arm has an excellent main cross-sectional axis that is significantly larger than a second cross-sectional axis. A wire-shaped sensor arm has a low weight and can be manufactured inexpensively and in a simple manner in different lengths. In addition, the sensor arm can be arranged/supported in a very compact (bent) manner in its mounting position. Furthermore, the length of the sensor arm can be provided variably to cover different distances to a measurement location depending on the actual dimension of the analysis location (e.g. toilet bowl, urinal, etc.) where the on-site analysis device is mounted.

According to another preferred further development of the subject matter of the invention, a winding roller is rotatably mounted in the circumferential direction on the holder of the device for on-site analysis, the winding roller having a circumferentially extending winding groove which is recessed in the radial direction of the winding roller and into which the sensor arm can be inserted and removed, the spacing means being set up and arranged to selectively enable or prevent the insertion and/or removal of the sensor arm into or from the winding groove, respectively.

The winding roller is a compactly designed component which accommodates the sensor arm in its storage position bent in the winding groove in a space-saving manner. Different working lengths of the sensor arm can be provided by the on-site analysis device depending on the winding capacity of the winding roller (i.e., depending on the diameter and depth of the winding groove), so that the on-site analysis device can be used flexibly and without special adaptations at different analysis locations, for example at toilet bowls of different sizes, urinals and the like.

Furthermore, the winding roller with its winding groove provides a space in which the sensor arm is compactly and securely received when spaced from those parts of the holder that may provide frictional contact in the direction of longitudinal displacement of the sensor arm. It is to be understood that the winding roller or the winding groove, in the sense of the invention, are not themselves such critical portions of the holder. It is true that during removal or insertion (unrolling/rolling) of the sensor arm from or into the winding groove, the sensor arm and the winding groove or inner walls thereof move relative to each other. However, this relative movement takes place in the radial direction of the winding roller, i.e. perpendicular to the longitudinal displacement direction of the sensor arm and thus not in the direction of the longitudinal displacement of the sensor arm, which the latter undergoes when being unwound from or wound onto the winding roller with respect to the holder or portions thereof. Finally, the spacing means ensures that the part of the sensor arm received in the winding groove cannot leave the winding groove in an uncontrolled manner and come into contact with the critical portions of the holder.

The winding roller also offers the advantage of an easy-to-implement drive for longitudinal displacement of the sensor arm, for example, in that the actuator rotationally drives the winding roller to push the sensor arm longitudinally, for example, into the measurement position and pull it back into the mounting position. During this process, the push and pull drive forces are efficiently transmitted longitudinally by the driven winding roller due to the tensile and compressive stiffness properties of the sensor arm already mentioned herein.

The holder may further comprise a housing in which the winding roller is rotatably received, but without necessarily being limited thereto. The holder as well as the housing are parts which are fixed and static in relation to the sensor arm. In this case, the selective locking of the winding groove effected by the spacing means ensures that the part of the sensor arm received in the winding groove is kept at a distance, for example, from inner walls of the housing. The inner walls are critical portions in the sense of the invention with respect to the longitudinal displacement direction of the sensor arm, since they could provide a frictional contact during the longitudinal displacement of the sensor arm between its mounting and its measuring position if the sensor arm were to contact the inner walls.

According to a further advantageous embodiment of the invention, the spacing means and/or at least one groove wall delimiting the winding groove is/are elastically deformable in such a way that, by selectively exerting a predetermined deformation force on the spacing means and/or on the groove wall, the winding groove is unobstructed for insertion or removal of the sensor arm and is otherwise obstructed. This creates a mechanism that is favorable in terms of design and by means of which the sensor arm can be held in the winding groove or released therefrom.

Furthermore, it is particularly advantageous if the predetermined deformation force can be applied solely by means of the sensor arm itself by exerting a predetermined tensile and/or thrust force on it, for example from the winding roller or the actuator driving it. This eliminates the need for an additional component for providing the deformation force, so that the device for on-site analysis is of simpler and more compact construction overall and is inexpensive to manufacture.

In an alternative embodiment of the invention, the holder comprises an engagement element that is operably engageable with the spacing means and/or with the at least one groove wall. In this case, the engagement element exerts the predetermined deforming force on the spacing means and/or on the groove wall. If the holder has, for example, a housing in which the winding roller is accommodated, the engagement element can, for example, be fastened to inner walls of the housing and thus act on the spacing means and/or the groove wall of the winding groove. The engagement element may likewise be attached to the holder.

According to a still further advantageous embodiment of the invention, the spacing means is at least one retaining claw attached to the one groove wall and extending with a free end in the direction of a further groove wall opposite the one groove wall. The at least one retaining claw (or retaining lip) thus bridges the open end of the winding groove on the outer circumference of the winding roller. Only one end of the retaining claw is fixedly connected to the one groove wall, for example formed integrally therewith. The other end of the retaining claw, which is directed towards the further groove wall, is a free end which can be movable, in particular with respect to this further groove wall, in order thereby to be able to selectively unblock or block the winding groove with regard to the removal or insertion of the sensor arm. For example, the retaining claw and/or at least one of the two groove walls can be designed to be elastically deformable in order to release or block the winding groove by applying a deformation force, as described for example above, for rolling the sensor arm onto or down from the winding roller, respectively.

In a further advantageous embodiment of the invention, a plurality of retaining claws are arranged in the circumferential direction of the winding groove, the retaining claws being fastened at one end in alternating sequence to the one groove wall and to the further groove wall opposite the one groove wall. The alternating arrangement of the retaining claws, which is symmetrical with respect to the winding groove, makes it possible to further improve the properties when winding and unwinding the sensor arm onto and from the winding roller.

Another advantageous further development of the subject matter of the invention provides for the spacing means to be a sinous spring or endless spiral (e.g., helical spring or the like) arranged in the winding groove and circumferentially surrounding the winding groove. The sinous spring, unlike the endless spiral, has a substantially two-dimensional sinous configuration, whereas the endless spiral has a three-dimensional helical configuration. It is to be understood that the sinous spring is arranged in the winding groove in such a way that the sinous crests and sinous troughs of the sinous arrangement always extend toward the opposing groove walls, in particular touching them at least partially. Likewise, the outside of the endless spiral can be at least partially in contact with the opposing groove walls. In any case, the sinous spring or the endless spiral limit the winding groove in the radial direction at the outer circumference of the winding roller, so that the radially outer end of the winding groove, which is open per se, is basically closed by the sinous spring or endless spiral and the part of the sensor arm arranged in the winding groove is held therein.

Both the sinous spring and the endless spiral, which may be in the form of a helical spring, for example, may be elastically deformable so that they selectively unblock or block the winding groove at their outer circumference, so that the part of the sensor arm accommodated in the winding groove is, on the one hand, held securely in the winding groove and, on the other hand, can nevertheless be guided out of the winding groove or brought into the winding groove in the desired manner by applying the deforming force described above.

A preferred further development of the subject matter of the invention provides for the sinous spring to be arranged so as to be longitudinally displaceable in its circumferential direction of travel relative to the winding groove, or for the endless spiral to be arranged rotatably about its longitudinal axis. For this purpose, the sinous spring or the endless spiral can be held in a suitably designed groove of the respective groove wall of the winding groove so as to be longitudinally displaceable or rotatable within the winding groove, in particular by the groove walls facing each other.

The longitudinally displaceable sinous spring enables the sensor arm to be guided out of the winding groove transversely through a sinous crest or sinous trough of the sinous spring. In other words, the sensor arm is threaded out of or into the winding groove transversely to the sinous spring. If the winding roller rotates in a certain direction of rotation to roll or unroll the sensor arm into or out of the winding groove, the sinous spring is displaced by a corresponding degree in the direction opposite to the direction of rotation of the winding roller, so that the location of the unthreading or threading point for the sensor arm essentially does not change relative to the holder. The sinous spring ensures at all times over the entire circumference of the winding roller that the part of the sensor arm mounted in the winding groove cannot come out of the winding groove in an uncontrolled manner. The sensor arm is always inserted into or removed from the winding groove only at the insertion or removal point specified by the sinous spring and fixed relative to the holder. This allows the sensor arm to be unrolled from the winding groove or rolled back into it without interference.

The endless spiral which can be rotated about its longitudinal direction of extension, for example in the manner of a spiral spring but without necessarily being limited thereto, makes it possible to accommodate the entire part of the sensor arm to be stored in the winding groove completely in the inner volume limited by the spiral arrangement. In this case, too, the sensor arm can be guided out of the inner volume of the endless spiral at a predetermined point, i.e. between two predetermined spiral turns. If the winding roller rotates in a certain direction of rotation for unwinding or winding the sensor arm out of or into the winding groove, the endless spiral rotates in such a way that the threading or unthreading point between the two spiral turns at which the sensor arm is guided out of the winding groove or the spiral inner volume does not change with respect to the holder. The sensor arm is always inserted into or removed from the winding groove at the insertion or removal point specified by the endless spiral which is fixed relative to the holder. This allows the sensor arm to be unwound from or rewound into the winding groove without interference.

According to a further preferred embodiment, a nominal groove width of the winding groove is defined in such a way that successive windings of the sensor arm can be arranged in the winding groove adjacent to one another only in the radial direction. The nominal groove width is the distance between two opposing groove walls bordering on the winding groove, which these have in the normal, free, unloaded state of the winding roller. This design prevents multiple windings of the sensor arm from being arranged in the axial direction of the winding roller. Thus, the nominal groove width of the winding groove can assume a value that is only slightly larger than a thickness of the sensor arm to be accommodated in the winding groove. Upwardly, the nominal groove width according to this embodiment is limited by twice the thickness of the sensor arm, that is, the nominal groove width is smaller than twice the thickness of the sensor arm to be accommodated in the winding groove. The merely radially adjacent arrangement of sections of the sensor arm in the winding groove additionally ensures that the windings of the sensor arm cannot cross each other when unwinding from the winding roller in the winding groove and thereby jam each other. This ensures a smooth pushing drive of the winding roller, in which the sensor arm is unwound from the winding groove by the pushing force applied by the winding roller, for example, in order to be shifted from the mounting position to the measuring position.

According to yet another further embodiment of the subject matter of the invention, the spacing means or, if another (e.g. first) spacing means is already provided, another (e.g. second) spacing means is a sheathing which at least partially envelops the sensor arm and receives it in a longitudinally displaceable manner. Here, an outer side of the sheathing is supported on the holder, preferably held thereon. Also, as described herein, the sheathing spaces the sensor arm from critical portions of the holder that may come in frictional contact with the sensor arm with respect to longitudinal displacement in the longitudinal direction of the sensor arm.

In a particularly preferred further embodiment, the sheathing has a Teflon material on its inner surface facing the sensor arm. This also includes the possibility that the sheath is formed entirely of a Teflon material. The Teflon material reduces friction between the sheathing and the sensor arm when the sensor arm is moved back and forth between the measurement position and the mounting position.

In another embodiment of the invention, the actuator rotationally drives the winding roller to move the sensor arm between its mounting position and its measurement position. Thus, during unwinding, the winding roller pushes the sensor arm from its mounting position to its measuring position. When winding up, the winding roller pulls the sensor arm back into the mounting position accordingly. The arrangement of the actuator as well as the coupling with the winding roller can be implemented in a space-saving and structurally simple manner.

According to an alternative embodiment, the actuator comprises a drive roller which cooperates with the sensor arm in a frictional manner in order to displace the latter between its extended measuring position and its retracted mounting position in the manner described herein, i.e. to push or pull it depending on the displacement direction.

According to a still further advantageous embodiment of the subject matter of the invention, a linear (e.g., thread-like) pulling means is attached to a portion of the sensor arm enveloped by the sheath and protrudes from a first open end of the sheath and an opposite second open end of the sheath. The actuator is arranged and configured to cooperate with the pulling means in such a way that, for longitudinal displacement of the sensor arm into the measuring position, it exerts (only) a traction force on a section of the pulling means protruding from the first open end of the sheath, and for longitudinal displacement of the sensor arm into the mounting position, it exerts (only) a traction force on a section of the pulling means protruding from the second end of the sheath. Thus, for each displacement movement of the sensor arm, the actuator always exerts a tensile force on only one of the two sections of the pulling means protruding from the sheathing.

The pulling means may be formed as a thread, that is, as a flexurally slack structure having a dominant one-dimensional extension and a uniformity in the longitudinal direction. The pulling means may be composed of one fiber (e.g. nylon thread) or of several fibers (textile, synthetic fibers and the like). The cross-section of the tensile means is not limited to substantially circular cross-sections, but may also be substantially ribbon-shaped.

The sections of the pulling means projecting from the sheathing in each case can be free ends, each of which is wound around a motor shaft of a drive motor (=actuator), for example, so that the actuator pulls either on one section or on the other section of the pulling means, depending on the direction of rotation of the motor shaft. It is also conceivable to design the pulling means as a closed pulling means loop, that is, the section protruding from the first end of the sheath is connected to the pulling means section protruding from the second end of the sheath. The actuator could act in a pulling manner on the pulling means via a drive wheel around which the pulling means is wound, in the manner described herein.

In another preferred embodiment of the invention, the on-site analysis device is housed in a housing which is a toilet seat attachable to a toilet bowl for receiving excrement. Thus, it is possible to attach the device for on-site analysis in a simple, aesthetically pleasing, safe and also extremely space-saving manner to different toilet bowls, which may, for example, have different sizes with correspondingly different distances between the holder and the measurement location.

A housing for the on-site analysis device to be provided in addition to the toilet seat is thus not required. To accommodate the on-site analysis device, the toilet seat can have, for example, a closable cavity or a recess open towards the outside, in which the device can be inserted and held securely and without great effort.

In an advantageous alternative embodiment, the device for on-site analysis has a housing in which the device is accommodated. The housing also has a holding means with which an attachment to a toilet bowl for receiving the excrements can be produced. In this case, the device for on-site analysis can be attached directly to the toilet bowl without the need to provide, for example, a special toilet seat as described in the preceding example of embodiment. Retrofitting an existing toilet facility with the on-site analysis device according to the invention can be carried out easily and inexpensively in this way. The holding means can, for example, provide a form-fitting fastening option (e.g. retaining clip) that can be plugged onto the toilet bowl, e.g. onto the bowl rim. Alternatively or additionally, the holding means may also provide a magnetically acting and/or clamping, substantially frictionally acting attachment/securing means between the toilet bowl and the housing of the on-site analysis device. For this purpose, the toilet bowl can have, for example, a recess or cavity into which the device can be inserted accordingly, but without necessarily being limited thereto. In this way, the device for on-site analysis can, for example, be flush with an outer surface of the toilet bowl or be accommodated essentially completely in the material of the toilet bowl, so that both a compact installation and an aesthetically pleasing overall impression can be achieved. A recess open towards an outer side of the toilet bowl offers in particular the advantage of being able to mount or dismount the device for on-site analysis on the toilet bowl without great effort.

Preferably, the holding means is designed in such a way that non-destructive separation of the device for on-site analysis from the toilet bowl is possible. In this way, the device for on-site analysis can also be provided and used only temporarily on a toilet facility if, for example, the control of excrement is only required for a limited period of time.

According to a still further advantageous embodiment of the invention, the device for on-site analysis comprises a self-sealing cartridge for providing several sample carriers which are accommodated in a cartridge housing. The sample carriers, e.g. indicator strips, are used to detect the excrements at the measurement location. An unused sample carrier is brought to the measuring location by means of the sensor arm and held there by the sensor arm. The cartridge housing can preferably be mounted near the holder of the sensor arm, so that a new sample carrier can be fed to the sensor arm at the beginning of each new analysis process as it leaves or shortly after it has left the mounting position of the sensor arm. The arrangement of the cartridge housing away from the measuring location additionally prevents unintended contamination of the sample carriers accommodated in the sample housing.

In general, the cartridge can have a memory chip on which sample carrier-relevant data, such as indication, production date, expiration date, batch number, number of sample carriers, etc., are stored. The memory chip can be designed to be writable in order to be able to update the number of remaining sample carriers after each sample carrier removal. The memory chip can be designed as an RFID chip, for example. The sample carrier cartridge can be mechanically clamped in its own housing or in a housing that accommodates the device for on-site analysis, and can additionally or alternatively be held in place by magnetic action.

In particular, the sample carrier cartridge may be replaceably retained on the on-site analysis device or a housing accommodating the same, so that an empty/old sample carrier cartridge may be replaced by a full/new one at any time.

The cartridge housing is formed by a first housing half and a second housing half, wherein the first housing half and the second housing half are held so as to be displaceable relative to one another between a closed position and a dispensing position and are biased into the closed position by a spring element. In the closed position, the sample carriers accommodated in the cartridge housing are shielded from an external environment, in particular substantially hermetically shielded, providing reliable protection of the sample carriers from contamination, moisture and the like. In the dispensing position, at least one of the sample carriers received in the cartridge housing can be removed from the cartridge housing.

Particularly preferably, at most only one sample carrier can be removed from the cartridge housing in the dispensing position, so that no further sample carrier can fall out of the cartridge housing unintentionally during sample carrier removal. The spring element biasing the housing parts into the closed position automatically ensures that the cartridge housing is closed again (hermetically) after each sample carrier removal.

A preferred further development of the invention further provides for the spring element to be mounted at one end on the first half of the housing and at the other end to press on a feed plunger which is displaceably mounted in the second half of the housing and presses the sample carriers (e.g. sample carrier stack) accommodated in the cartridge housing against a housing wall of the second half of the housing. In the area of the sample carrier of the sample carrier stack resting against the housing wall of the second housing half, a dispensing window is provided in the second housing half for dispensing the sample carrier resting against the housing wall from the cartridge housing. In the closed position, the dispensing window is closed by a wall portion of the first half of the housing, in particular to substantially hermetically shield the sample carriers stored in the cartridge housing from the external environment, as already described in the preceding example of embodiment. In the dispensing position, the dispensing window is uncovered to removably provide the sample carrier abutting against the housing wall of the second housing half from the cartridge housing through the dispensing window. An opening of the dispensing window is preferably dimensioned such that removal of only one single sample carrier is possible.

According to a still further embodiment of the invention, an entrance window is provided in the first housing half of the cartridge housing in the region of the sample carrier of the sample carrier stack resting against the housing wall of the second housing half, which entrance window is closed by a wall section of the second housing half in the closed position of the cartridge housing. In the output position of the cartridge housing, the entrance window is uncovered, as is the dispensing window described above. The entrance window is arranged in such a way that by means of insertion of an ejection plunger external to the cartridge housing through the entrance window, the sample carrier resting against the housing wall of the second housing half can be pushed out of the cartridge housing through the dispensing window.

According to another aspect of the invention, a self-sealing cartridge for providing sample carriers, such as indicator strips, for an apparatus for on-site analysis of excrements according to the invention comprises a cartridge housing receiving the sample carriers. The cartridge housing is formed by a first housing half and a second housing half, wherein the first housing half and the second housing half are held so as to be displaceable relative to one another between a closed position and a dispensing position and are biased into the closed position by means of a spring element. In the closed position, the sample carriers accommodated in the cartridge housing are shielded from an external environment, in particular substantially hermetically shielded, which reliably protects the sample carriers from contamination with excrement, from moisture and the like. In the dispensing position, at least one of the sample carriers accommodated in the cartridge housing is removable from the cartridge housing.

Particularly preferably, only one single sample carrier at most can be removed from the cartridge housing in the dispensing position, so that no further sample carrier can fall out of the cartridge housing unintentionally during sample carrier removal. The spring element, e.g. a helical spring, which biases the housing parts into the closed position, automatically ensures that the cartridge housing is closed again (hermetically) after each sample carrier removal.

The cartridge can preferably have a memory chip on which sample carrier-relevant data, such as indication, production date, expiration date, batch number, number of sample carriers, etc., are stored. The memory chip can be designed to be writable in order to be able to update the number of remaining sample carriers after each sample carrier removal. The memory chip can, for example, be designed as an RFID chip.

It should be noted that with respect to cartridge-related definitions of terms as well as the effects and advantages of cartridge-related features, full reference is made to the disclosure herein of definitions, effects and advantages with respect to the on-site analysis device of the invention.

Disclosures herein relating to the on-site analysis device of the invention may also be used, mutatis mutandis, to define the sample carrier cartridge of the invention, unless expressly excluded. Likewise, disclosures herein relating to the cartridge according to the invention may be used, mutatis mutandis, to define the on-site analysis device according to the invention, unless this is expressly excluded. Consequently, a repetition of explanations of mutatis mutandis identical features, their effects and advantages of a more compact description can be dispensed with, without such omissions being construed as a limitation.

An advantageous embodiment of the subject matter of the invention provides for the spring element to be supported at one end by the first housing half and to press with the other end a feed plunger displaceably mounted in the second housing half. The feed plunger in turn presses the sample carriers (e.g. sample carrier stack) held in the cartridge housing against a housing wall of the second housing half. In the area of the sample carrier of the sample carrier stack resting against the housing wall, a dispensing window is provided in the second housing half for dispensing the sample carrier resting against the housing wall from the cartridge housing. In the closed position, the dispensing window is closed by a wall section of the first half of the housing in order to shield the sample carriers stored in the cartridge housing, in particular, essentially hermetically from the external environment. In the dispensing position, the dispensing window is uncovered to removably provide the sample carrier resting against the housing wall of the second housing half from the cartridge housing through the dispensing window. An opening of the dispensing window is preferably dimensioned in such a way that the removal of only one sample carrier is possible.

Further according to an advantageous embodiment of the subject matter of the invention, an entrance window is arranged in the first housing half of the cartridge in the region of the sample carrier of the sample carrier stack resting against the housing wall of the second housing half, which entrance window is closed in the closed position by a wall section of the second housing half. This again ensures a substantially hermetic shielding of the sample carriers received in the cartridge housing. In the dispensing position, the entrance window is uncovered. The entrance window is arranged such that by insertion of an ejection plunger external to the housing through the entrance window, the sample carrier of the sample carrier stack resting against the housing wall of the second housing half can be pushed out of the cartridge housing through the dispensing window.

According to a still further aspect of the invention, a toilet device according to the invention for analyzing excrements, in particular human excrements, comprises a collecting device for collecting the excrements. Attached to the collection device is a device for on-site analysis of the excrement according to one of the embodiments disclosed herein.

With respect to the toilet device according to the invention, it should also be noted that for toilet device-related definitions of terms and effects and advantages of device-related features, full reference is made to the disclosure herein of definitions, effects and advantages with respect to the on-site analysis device according to the invention and the self-sealing cartridge according to the invention. Disclosures herein relating to the on-site analysis device and cartridge according to the invention may also be referred to mutatis mutandis to define the toilet device according to the invention, unless expressly excluded. Likewise, disclosures herein relating to the toilet device according to the invention may be used, mutatis mutandis, to define the on-site analysis device according to the invention and the sample carrier cartridge according to the invention, unless this is expressly excluded. Consequently, a repetition of explanations of analogously identical features, their effects and advantages of a more compact description can be dispensed with, without such omissions being construed as a limitation.

In an advantageous further development, the toilet device according to the invention can further comprise a measuring device for measuring the geometry of a surface of the collection device, which comprises at least one light source (e.g. LED, laser, etc.) for generating light, at least one projection device for projecting the light in a predetermined geometric pattern onto the surface of the collection device, at least one light-sensitive sensor, in particular a camera, for recording the geometric pattern imaged on the surface of the collection device, and an electronic control device, for example a microcontroller. The control device is further arranged to determine the surface geometry (topology) of the collection device from a deviation of the recorded geometric pattern imaged on the surface of the collection device from the originally projected geometric pattern. By knowing the exact surface geometry of the collection device, it is possible to automatically determine, for example, an optimal measurement location for accurate excrement analysis, which is approached by the sensor arm of the device for on-site analysis.

In addition, further parameters for the analysis of the excrements can be recorded and evaluated by means of the evaluation described above in order to expand and/or further specify the analysis results. For example, a volumetric flow rate of a urine stream or a runoff film on the surface of the collection device can be detected.

Lenses, apertures, or diffractive optical elements (DOE) may further be provided for projecting the geometric pattern onto the surface of the collection device.

The geometric pattern to be projected may be formed of lines, dots, dot-dash combinations, crosses, etc.

According to an advantageous embodiment of the invention, two cameras are provided and arranged to record the geometric pattern imaged on the surface of the collection device from different recording directions. This enables a very precise determination of the surface geometry (topology) of the collecting device. For example, triangulation methods known per se can be used for this purpose.

In a further advantageous embodiment, the toilet device has two projection devices arranged to project the predetermined geometric pattern onto the surface of the collection device from different projection angles. This allows the surface geometry (topology) of the collection device to be alternatively or additionally recorded more precisely.

Particularly preferably, according to a further embodiment, the collecting device of the toilet device is a toilet bowl to which a toilet seat is attached. In this case, the device for on-site analysis of the excrement is accommodated in the toilet seat. Thus, the device for on-site analysis can be attached to different toilet bowls in an aesthetically pleasing, extremely space-saving and safe manner. For example, the toilet bowls may have different sizes with accordingly different distances between the device for on-site analysis or the holder of the sensor arm and the measuring location, which can be bridged by the sensor arm in the manner described herein. An additional housing for the on-site analysis device need not be provided on the toilet device, as the toilet seat already provides the housing therefor.

Alternatively, the device for on-site analysis can also be accommodated in its own housing, in which case the housing can in turn have a holding means with which attachment to the toilet bowl of the toilet device can be established. In this way, for example, an existing toilet facility can be easily and inexpensively retrofitted. The holding means can, for example, provide a form-fitting fastening option (e.g. retaining clip) that can be plugged on or inserted into the toilet bowl, e.g. onto the bowl rim or into a recess/cavity in the bowl rim. Alternatively or additionally, the holding means may provide a magnetic and/or frictional attachment/securing between the toilet bowl and the housing of the on-site analysis device. The on-site analysis device may, for example, be flush with an outer surface of the toilet bowl or be substantially completely contained within the toilet bowl, so that both a compact mounting and an aesthetically pleasing overall appearance can be achieved. A recess open towards an outer side of the toilet bowl offers in particular the advantage of being able to assemble or disassemble the device for on-site analysis on the toilet bowl without any great effort.

Preferably, the holding means is designed in such a way that non-destructive separation of the device for on-site analysis from the toilet bowl is possible. In this way, the device for on-site analysis can also be provided and used only temporarily on a toilet device if, for example, the control of excrement is desired only for a limited period of time.

According to a still further advantageous embodiment, the collection device of the toilet device is a toilet bowl with a toilet seat attached thereto, wherein the measuring device for measuring the geometry of the surface of the collection device is accommodated in the toilet seat. In other words, the toilet seat forms the housing of the measuring device. Particularly preferably, the toilet seat for receiving the measuring device can also simultaneously form the housing of the device for on-site analysis, as already described above, but the invention is not necessarily limited thereto. In any case, the measuring device can thus be attached to different toilet bowls in an aesthetically pleasing, space-saving and safe manner.

According to a further aspect of the invention, a toilet seat according to the invention for a toilet device having a toilet bowl for collecting excrement comprises a device received in the toilet seat for on-site analysis of the excrement collected in the toilet bowl according to one of the embodiments disclosed herein. The toilet device may in turn be configured according to any of the embodiments of toilet devices according to the invention disclosed herein.

Accordingly, full reference is made to the disclosure herein of definitions, effects and advantages with respect to the on-site analysis device of the invention and the toilet device of the invention with respect to toilet seat-related definitions and the effects and advantages of toilet seat-related features. Disclosures herein relating to the on-site analysis device of the invention and the toilet device may also be used, mutatis mutandis, to define the toilet seat of the invention, unless this is expressly excluded. Likewise, disclosures herein relating to the toilet seat according to the invention may also be used, mutatis mutandis, to define the on-site analysis device and toilet device according to the invention, unless this is expressly excluded. Consequently, a repetition of explanations of analogously identical features, their effects and advantages of a more compact description can be dispensed with, without such omissions being construed as a limitation.

Further features and advantages of the invention will be apparent from the following description of non-limiting examples of embodiment of the invention, which will be explained in more detail below with reference to the drawing. In this drawing, the following are schematically shown:

FIG. 1 is a perspective view of both an example of embodiment of a device for on-site analysis of excrement according to the invention and an example of embodiment of a toilet device for analysis of excrement according to the invention;

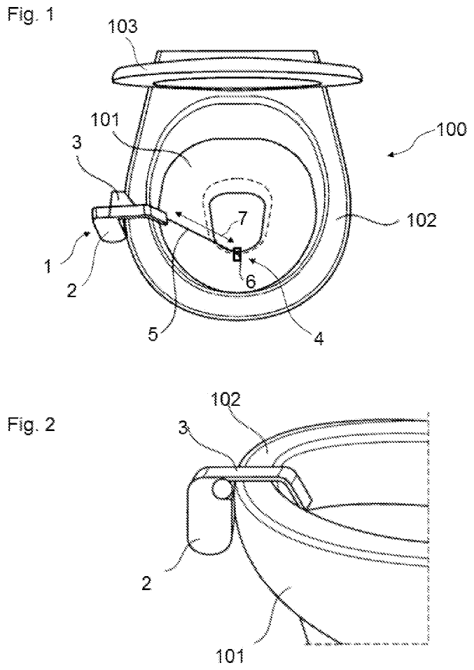

FIG. 2 is a partial perspective view of the device for on-site analysis and of the toilet device of FIG. 1;

FIG. 3 is a perspective view of the device for on-site analysis from FIG. 1 in detail;

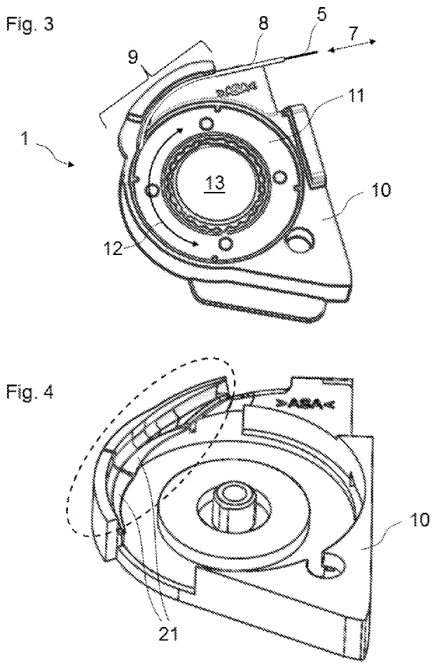

FIG. 4 is a perspective view of a part of the device for on-site analysis from FIG. 3;

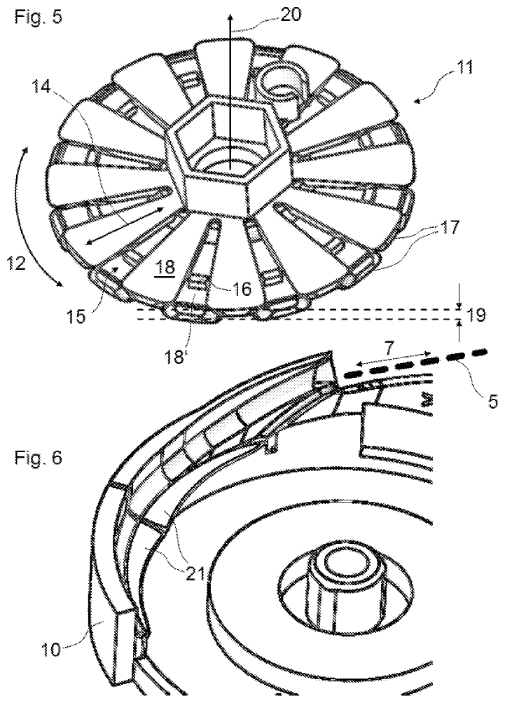

FIG. 5 is a perspective view of another part of the device for on-site analysis from FIG. 3;

FIG. 6 is an enlarged partial perspective view of the part of the device for on-site analysis from FIG. 4;

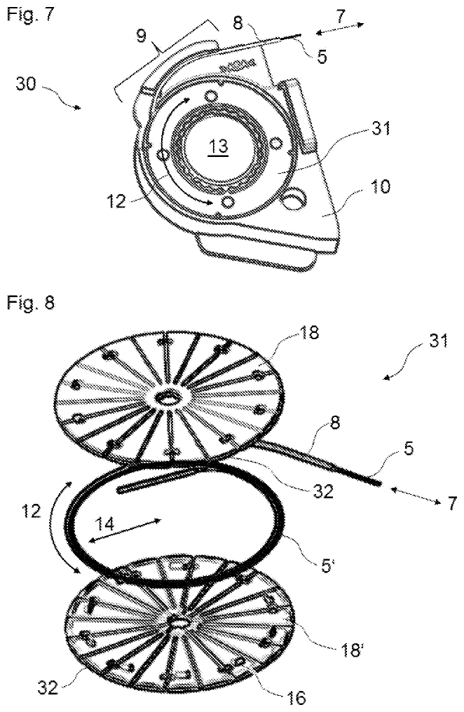

FIG. 7 is a perspective view of a further example of embodiment of a device for on-site analysis according to the invention;

FIG. 8 is a perspective exploded view of several parts of the device for on-site analysis of FIG. 7;

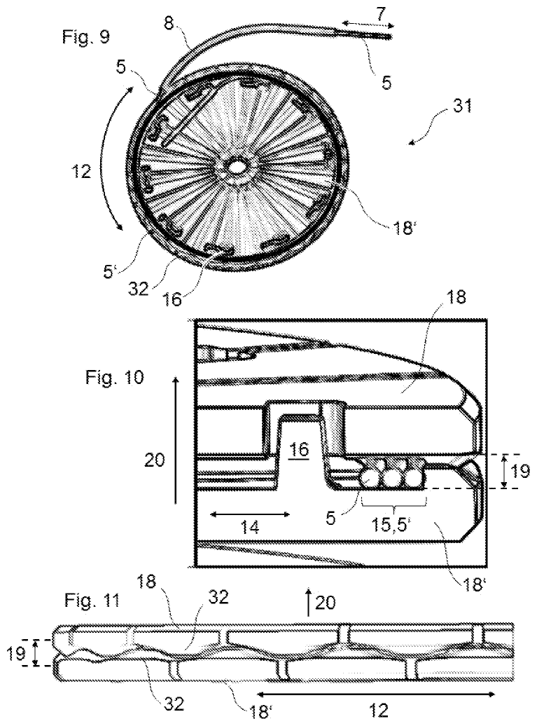

FIG. 9 is a top view of a part of the device for on-site analysis of FIG. 8;

FIG. 10 is an enlarged perspective cross-sectional view of the parts of the on-site analysis device of FIG. 8 in the assembled state;

FIG. 11 is an enlarged perspective partial view of the parts of the device for on-site analysis from FIG. 8 in the assembled state;

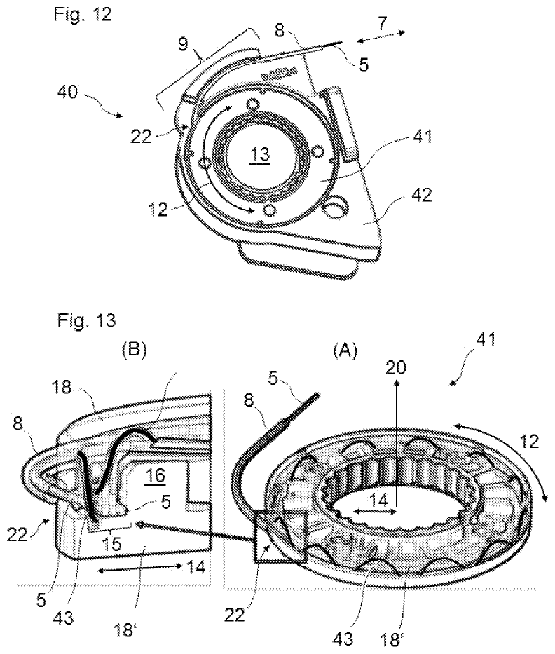

FIG. 12 is a perspective view of yet another embodiment of a device for on-site analysis according to the invention;

FIG. 13 shows, in view A, a perspective view of a portion of the on-site analysis apparatus of FIG. 12, and in view B, an enlarged cross-sectional perspective view of a portion of the on-site analysis apparatus of view A;

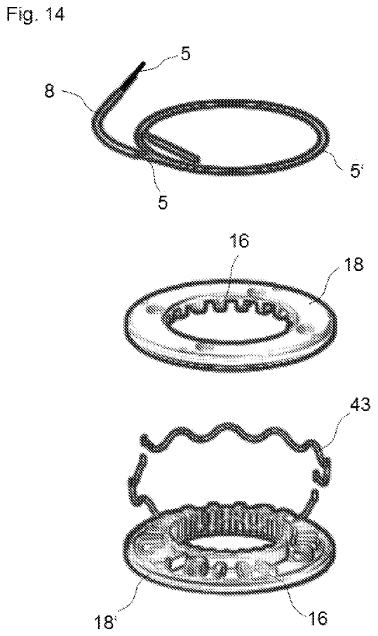

FIG. 14 is an exploded perspective view of several parts of the on-site analysis device of FIG. 12;

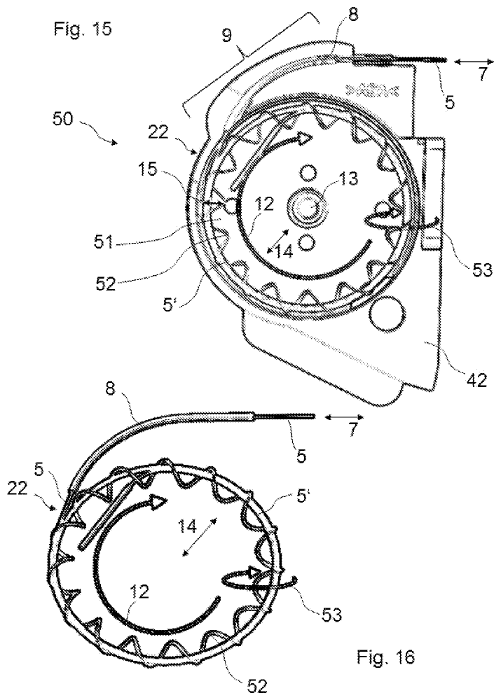

FIG. 15 is a top view of yet another example of embodiment of a device for on-site analysis according to the invention;

FIG. 16 is a top view of parts of the device for on-site analysis from FIG. 15;

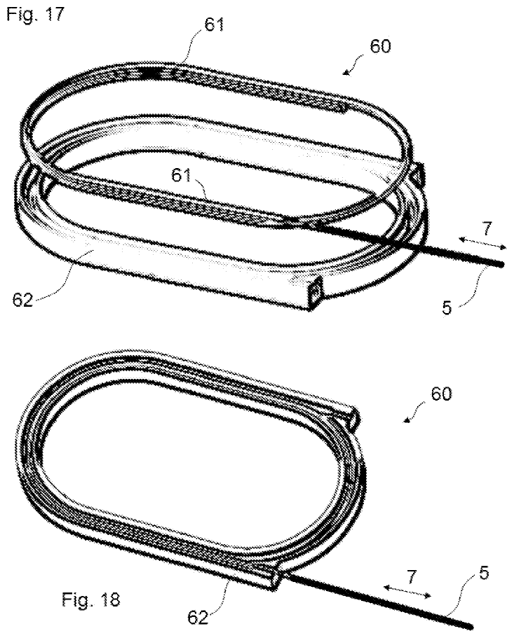

FIG. 17 is a perspective exploded view of still another example of embodiment of a device for on-site analysis according to the invention;

FIG. 18 is a perspective view of the device for on-site analysis of FIG. 17 in the assembled state;

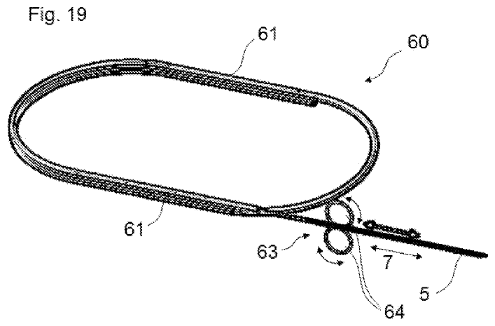

FIG. 19 is a further perspective view of parts of the device for on-site analysis of FIG. 17;

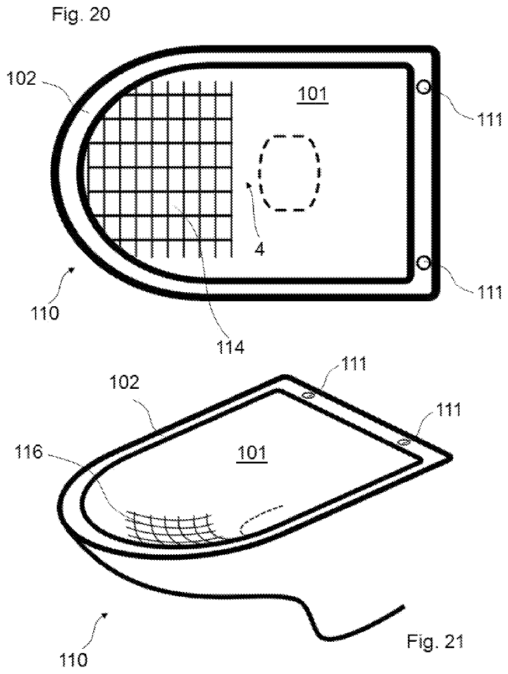

FIG. 20 is a top view of an example of embodiment of a toilet device according to the invention;

FIG. 21 is a perspective view of the toilet device of FIG. 20;

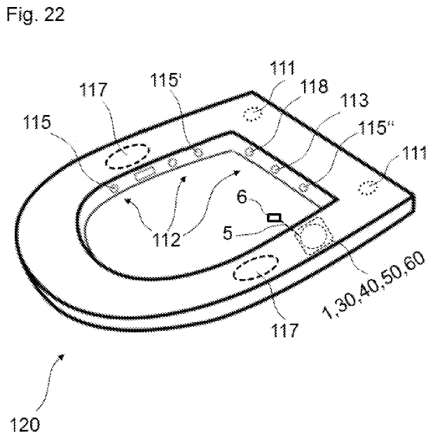

FIG. 22 is a perspective view of an example of embodiment of a toilet seat according to the invention;

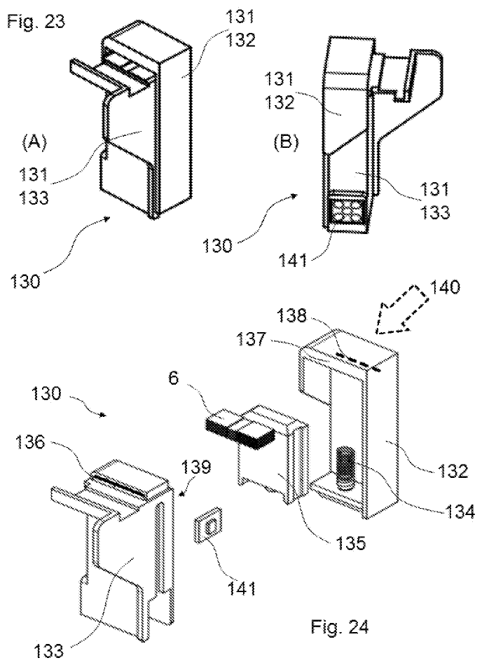

FIG. 23 shows two different perspective views A and B of an example of embodiment of a self-sealing cartridge for sample carriers according to the invention;

FIG. 24 is a perspective exploded view of the cartridge of FIG. 23;

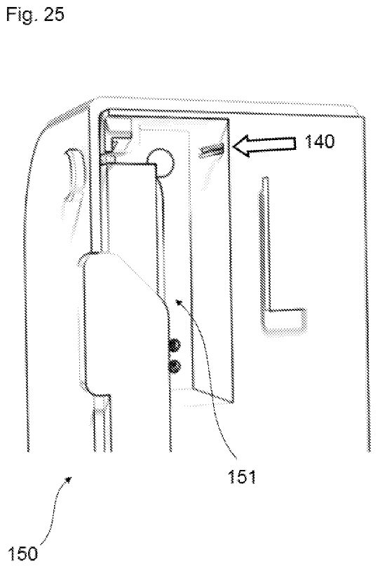

FIG. 25 is a perspective view of an example of a sample carrier dispensing device for receiving the cartridge of FIG. 23; and

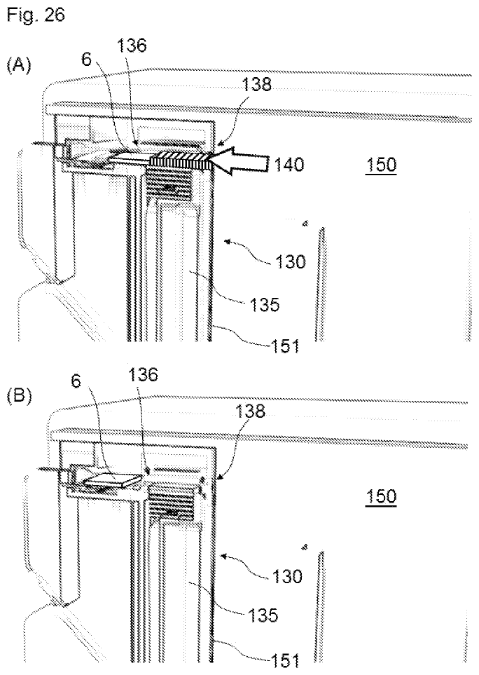

FIG. 26 is a perspective view A of the cartridge from FIG. 23 accommodated in the sample carrier dispensing device from FIG. 25 in a dispensing position and a perspective view B of the sample carrier dispensing device from view A in a closed position.

In the various figures, parts that are equivalent in terms of their function are always provided with the same reference numbers; therefore they are generally described only once.

FIG. 1 is a perspective view of both an embodiment of a device 1 for on-site analysis of excrement according to the invention and an example of embodiment of a toilet device 100 for analysis of excrement according to the invention. FIG. 2 is a partial perspective view of the device 1 and the toilet device 100 of FIG. 1. Both FIGS. 1 and 2 are referred to below.

FIGS. 1 and 2 show that the toilet device 100 for analyzing excrement, in particular human excrement, has a collecting device 101 (in this case in the form of a toilet bowl) for collecting the excrement. The device 1 for on-site analysis of the excrements is attached and fastened to the collecting device 101. In the present example, the device 1 is attached to an edge 102 of the toilet bowl 101. In the example shown, the fastening is effected by means of a holding means 3 provided on a housing 2, in which the device 1 is accommodated, which in the present case is designed as a retaining clip which can be positively plugged onto the toilet bowl or the bowl rim 102, without, however, necessarily being limited thereto. For example, the toilet bowl may also have a recess or cavity (both not shown) into which the device can be inserted accordingly. For example, the on-site analysis device may be flush with an outer surface of the toilet bowl, or may be received substantially entirely within the material of the toilet bowl. In FIG. 1, the toilet device 100 has been retrofitted with the device 1. In order to improve the stability of the attachment of the device 1 to the bowl rim 102, a magnetic and/or frictional securing means may also be provided between the toilet bowl 101 and the housing 2 of the device 1 (not shown) in addition to the retaining means 3. In any case, in the example shown in FIG. 1, the device 1 can be separated from the toilet bowl 101 in a non-destructive manner and without great effort.

Furthermore, the toilet device 100 in FIG. 1 comprises a toilet seat 103 tiltably attached to the toilet bowl 101 and known per se.

The exemplary device 1 for on-site analysis of excrement shown in FIG. 1 has a sensor device (not shown in detail), by means of which an excrement sample can be detected at a measuring location 4 and at least partially analyzed. In FIG. 1, a sensor arm 5 can be seen, which in the present case is elastically bendable and wire-shaped and can be mounted at least partially bent on a holder (not shown) accommodated in the housing 2. The sensor arm 5 serves to hold a sample carrier 6, for example an indicator strip, for detecting the excrement at the measuring location 4 after the sensor arm 5 has brought the sample carrier 6 there. FIG. 1 thus depicts a measuring position of the sensor arm 5, in which the latter, extended from the housing 2, bridges a distance from the holder, which cannot be recognized and is accommodated in the housing 2, to the measuring location 4. A position of the sensor arm 5 retracted from the measuring location 4 and at least partially bent on the holder is referred to as the mounting position (not shown). In the mounting position, the sensor arm 5 is at least partially, preferably completely, received in the housing 2. The sensor arm 5 is longitudinally displaceable (longitudinal direction 7) between the mounting position and the measuring position, the displacement being effected by an actuator which is not shown and which is likewise accommodated in the housing 2 in the present case.

It is to be understood that the sensor device may have further components in addition to the sensor arm 5 shown in FIGS. 1 and 2, even if they are not explicitly shown in the present case. Thus, according to the invention, the sensor device is set up for sensory detection and at least partial analysis of an excrement sample detected by the sample carrier 6. For this purpose, the sensor device may comprise at least one sensor which is not shown, preferably an entire sensor unit comprising a plurality of identical or different sensors (also not shown). For example, the sensor unit may comprise a color sensor for detecting a color change of the sample carrier, whereby a first analysis according to the invention is already performed. Other sensors may comprise, for example, touch sensors, ultrasonic sensors, temperature sensors, pressure sensors and the like, but are not necessarily limited thereto.

Advantageously, the sensor device and/or an additional device-external analysis unit which is not shown and to which the device-internal sensor device can transmit sensor data, preferably wirelessly, has a suitable electronic computing and control unit, for example a microprocessor, microcontroller and the like, and a corresponding electronic memory unit, for example ROM, RAM, flash memory, etc., for carrying out the analysis.

FIG. 3 is a perspective view of the device 1 for on-site analysis of FIG. 1 in detail without the housing 2 of FIGS. 1 and 2. The sensor arm 5 can be seen, which in the present example of embodiment is guided longitudinally displaceably along a bending section 9 of the sensor arm 5 in sections by a sheathing 8, without restriction thereto, for example a sheathing which is Teflon-coated on the inside or is formed entirely from a Teflon material. The sheath 8 can in principle be understood as one of several possible spacing means according to the invention, as will be explained in more detail below.

Furthermore, FIG. 3 shows a holder 10. On this holder, the sensor arm 5 can be mounted at least partially bent and in its retracted mounting position, as shown for example in FIG. 3, it is actually mounted at least partially bent.

In FIG. 3, it can be seen that a winding roller 11 is rotatably mounted on the holder 10 in its circumferential direction 12. In the present example, the winding roller 11 is rotatably drivable by the actuator not shown here, for example an electric motor, in order to displace the sensor arm 5 between its mounting position and its measuring position in the longitudinal direction 7. The actuator drives the winding roller 11, for example, via a drive shaft 13, onto which the winding roller 11 can be positively attached via suitable toothing, but without necessarily being limited thereto.

FIG. 4 shows a perspective view of the holder 10 of the device 1 for on-site analysis from FIG. 3 without the winding roller 11. A section of the holder 10 marked by a dashed line in FIG. 4 will be discussed in more detail in the description of FIG. 6 below.

FIG. 5 is a perspective view of the winding roller 11 of the on-site analysis device 1 of FIG. 3. It can be seen that the winding roller 11 has a winding groove 15 extending circumferentially (circumferential direction 12) and recessed in the radial direction 14 of the winding roller 11. The sensor arm 5 can be inserted into and removed from the winding groove 15. In the radial direction 14, the winding groove 15 is presently limited on the inside by respective limiting webs 16 and on the outside by retaining claws 17, which are provided at radially exterior free ends of opposing upper and lower groove walls 18, 18′, in particular presently formed integrally therewith, but without necessarily being limited thereto. FIG. 5 shows that in the winding roller 11 shown here the upper and lower groove walls 18, 18′ are arranged spaced apart from one another in a lamellar manner in the circumferential direction 12, the retaining claws 17 being fastened in alternating sequence at one end to the one upper groove wall 18 and at the other lower groove wall 18′ opposite the upper groove wall 18.

Preferably, but without necessarily being limited thereto, a nominal groove width 19 of the winding groove 15, which essentially corresponds to the free spacing of the opposite groove walls 18, 18′ in the axial direction 20 of the winding roller 11, is fixed in such a way that successive windings of the sensor arm 5 can be arranged in the winding groove 15 adjacent to one another only in the radial direction 14. That is, multiple windings of the sensor arm 5 within the winding groove 15 cannot be arranged adjacent to each other in the axial direction 20 of the winding roller 11 during winding due to the groove width 19 being substantially adapted to the thickness of the sensor arm 5.

The retaining claws 17 are spacing means according to the invention. That is, the retaining claws 17 are adapted and arranged to selectively allow or prevent the insertion and/or removal of the sensor arm 5 into and/or from the winding groove 15. Accordingly, the retaining claws 17 extend substantially across the groove width 19 to cover the winding groove 15 at the outer circumference of the winding roller 11 and thereby flexibly close it. Consequently, the sensor arm 5 inserted into the winding groove 15 is always kept at a distance, by the spacing means 17, from those parts of the holder 10 relative to which the sensor arm 5 moves in the direction of its longitudinal displacement 7 between its mounting position and its measuring position.

In the winding roller 11 shown in FIG. 5, the groove walls 18, 18′ bordering on the winding groove 15 are elastically deformable substantially in the axial direction 20 in such a way that by selective application of a predetermined deforming force on the spacing means or, in the present case, the retaining claws 17, the winding groove 15 is uncovered, and otherwise obstructed in that the retaining claws 17 are moved apart by the deforming force and the groove walls 18, 18′ are elastically bent up in the process.

It is to be understood that, although the winding roller 11 and/or the winding groove 15 may be conceived as a part of the holder 10, they are not themselves, in the sense of the invention, a critical part of the holder 10 with regard to frictional contact with the sensor arm 5 during displacement of the sensor arm 5 in the longitudinal direction 7 between its mounting position and its measuring position. It is true that during removal or insertion (unwinding/winding) of the sensor arm 5 from or into the winding groove 15, the sensor arm 5 and the winding groove 15 or the groove walls 18, 18′ thereof move in relation to each other. However, this relative movement takes place substantially exclusively in the radial direction 14 of the winding roller 11, that is, perpendicularly to the longitudinal displacement direction 7 of the sensor arm 5 and thus not in the direction of the longitudinal displacement 7 of the sensor arm 5 which the latter undergoes during unwinding and winding from and onto the winding roller 11 with respect to the holder 10 or parts thereof. The spacing means 17 ensure that the part of the sensor arm 5 received in the winding groove 15 cannot leave the winding groove 15 in an uncontrolled manner and come into contact with the critical parts of the holder 10.

FIG. 6 represents an enlarged perspective partial view of the holder 10 of the device 1 for on-site analysis from FIG. 4. It can be seen that the holder 10 has an engagement element 21 which can be brought into operative engagement with the spacing means or the retaining claws 17 of the winding roller 11 of FIG. 5 and which exerts the predetermined deformation force on the retaining claws 17 and correspondingly on the groove walls 18, 18′ connected thereto. As can be gathered from FIG. 6, the engagement element 21 in the example shown is attached to the holder 10, in this case formed integrally with the holder 10. The engagement element 21 projects in the form of a web or lip from the outside in the radial direction 14 between the retaining claws 17 of the winding roller 11 when the winding roller 11 is mounted on the holder 10. When the sensor arm is rolled onto or off the winding roller 11, the engagement of the engagement element 21 opening the winding groove 15 occurs directly at a point on the holder 10 at which the sensor arm 5 is fed to or leaves the winding roller 11, as illustrated in FIG. 6 by a depicted short dashed section of the sensor arm 5.

FIG. 7 illustrates a perspective view of a further embodiment of a device 30 for on-site analysis according to the invention. Essentially, the components of the device 30 shown correspond to the components of the device 1 of FIG. 3, except for a modified design of a winding roller 31.

FIG. 8 is a perspective exploded view of the winding roller 31 with the sensor arm 5 of the device 30 for on-site analysis from FIG. 7. In FIG. 8, the sensor arm 5 is shown in its storage position, in which a plurality of windings 5′ of the sensor arm 5 are stored bent in the winding groove of the winding roller 31. As can be seen further, the opposing groove walls 18, 18′ of the winding roller 31 are still arranged in a lamellar manner in the circumferential direction, but the distance between individual adjacent groove walls 18 or 18′ is selected to be significantly smaller than in the winding roller 11 of the device 1 of FIG. 5.

In the example shown, the winding roller 31 has retaining claws 32 which, however, in contrast to the retaining claws 17 of FIG. 5, which are essentially trapezoidal in the circumferential direction 12, are undulating in the circumferential direction 12. This makes it easier to insert the sensor arm 5 into or remove it from the winding groove 15 using less force. The undulating shape of the retaining claws 32 can be clearly seen in FIG. 11. This shape of the retaining claws 32 allows the predetermined deforming force for opening the winding groove 5 to be applied solely by means of the sensor arm 5, by applying a predetermined pulling and/or pushing force to the sensor arm 5 as it is unwound from or wound onto the winding roller 31. Thus, the holder 10 of the device 30 can dispense with the engagement element 21 of the device 1 and, consequently, can be of even simpler construction. However, the engagement element 21 may also be provided in the holder 10 of the device 30.

FIG. 9 is a top view of the lower groove wall 18′ with the sensor arm 5 of the device 30 for on-site analysis from FIG. 8 arranged thereon.

FIG. 10 shows an enlarged perspective cross-sectional view of the upper and lower groove walls 18, 18′ of the device 30 for on-site analysis from FIG. 8 in an assembled state. It can be clearly seen that the groove width 19 is dimensioned in such a way that individual windings 5′ of the sensor arm 5 can only lie adjacent to one another in the radial direction 14, but not in the axial direction 20.

FIG. 11 shows an enlarged perspective partial view of the outer circumference of the device 30 for on-site analysis from FIG. 8 in the assembled state. The wave- or hump-shaped configuration of the interlocking retaining claws 32 spanning the groove width 19 along the outer circumference 12 can be clearly seen.

FIG. 12 is a perspective view of yet another example of embodiment of a device 40 for on-site analysis according to the invention. The main difference between the device 40 and the device 1 of FIG. 3 is a different embodiment of a winding roller 41, which is rotatably mounted on a holder 42.

The holder 42 does not have an engagement element 21 like that of the holder 10 of the device 1 of FIG. 4.

FIG. 13 depicts in view A a perspective view of a portion of the winding roller 41 of the on-site analysis device 40 of FIG. 12, and in view B an enlarged cross-sectional perspective view of a portion of the winding roller 41 of view A.

In this example, the winding roller 41 has, as a spacing means, a sinous spring 43 arranged in the winding groove 15 and circumferentially surrounding the winding groove 15. Alternatively, the sinous spring 43 could also be formed as an endless spiral or helical spring (not shown).

It can be seen that the sinous spring 43 is arranged in the winding groove 15 in such a way that its wave crests and wave troughs are always directed towards the opposing groove walls 18, 18′. This holds the part of the sensor arm 5 arranged in the winding groove 15 inside the winding groove 15. The sinous spring 43 can be elastically deformable so that it can selectively uncover or block the winding groove 15 at its outer circumference 12. In particular, by applying the predetermined deforming force described herein, selective opening of the winding groove 15 otherwise blocked by the sinous spring 43 can be effected. This deforming force may be generated, for example, by the thrust and compression force applied on the sensor arm 5 during longitudinal displacement.

Alternatively, the sinous spring 43 can also be arranged and held so as to be longitudinally displaceable in its circumferential direction 12 of extension relative to the winding groove 15. For this purpose, the sinous spring 43 can be held, for example, in its own circumferentially extending sinous spring groove formed in the upper and lower groove walls 18, 18′, respectively. A longitudinally displaceable sinous spring 43 enables the sensor arm 5 to be guided outwardly out of the winding groove 15 transversely through a wave crest or wave trough of the sinous spring 43, as can be clearly seen in view B of FIG. 13. The sensor arm 5 is threaded out of or into the winding groove 15 transversely to the sinous spring 43. If the winding roller 41 rotates in a certain direction of rotation to unwind or rewind the sensor arm 5 from or into the winding groove 15, the sinous spring 43 is displaced by a corresponding amount in the direction opposite to the direction of rotation of the winding roller 41, so that a location 22 of the unthreading or threading point for the sensor arm 5 in relation to the holder 42 substantially does not change. At all times, the sinous spring 43 ensures along the entire circumference 12 of the winding roller 41, that the part of the sensor arm 5 supported in the winding groove 15 cannot get out of the winding groove 15 in an uncontrolled manner. The sensor arm 5 is always inserted into or removed from the winding groove 15 only at the insertion or removal point 22 specified by the sinous spring 43 and fixed relative to the holder 42. This allows the sensor arm 5 to be unwound from the winding groove 15 or rewound into it without interference.

FIG. 14 is a perspective exploded view of the winding roller 41 of the device 40 for on-site analysis from FIG. 12.

FIG. 15 is a top view of a still further example of embodiment of a device 50 for on-site analysis according to the invention. The essential difference between the device 50 and the device 40 of FIG. 12 is a winding roller 51, in which the spacing means is an endless spiral 52 arranged in the winding groove 15 and circumferentially surrounding the winding groove 15, which spiral 52 can be designed, for example, in the manner of a helical spring.

It is to be understood that the outer side of the endless spiral 52—similar to the sinous spring 43 of the device 40 of FIGS. 12 and 13—may be at least partially in contact with the opposing groove walls 18, 18′. In any case, the endless spiral 52 limits the winding groove 15 in the radial direction 14 at the outer circumference of the winding roller 51, so that the radially outer end of the winding groove 15, which is open per se, is basically closed by the endless spiral and the part of the sensor arm 5 arranged in the winding groove 15 is held therein.

In the present embodiment of the device 50, the endless spiral 52 is arranged to be rotatable about its longitudinal axis corresponding to the circumferential direction 12 (rotational direction 53). For this purpose, the endless spiral 52 can be rotatably held therein in a correspondingly configured groove of the respective groove walls 18, 18′ of the winding groove 15. Furthermore, in the example of embodiment shown, the endless spiral 52, which is rotatable about its longitudinal direction of extension, completely accommodates the part 5′ of the sensor arm 5 to be supported in the winding groove 15 in the inner volume enclosed by the spiral arrangement. Also in this case, the sensor arm is guided outwardly from the interior of the endless spiral 52 at a predetermined point 22, that is, between two predetermined spiral windings. If the winding roller 51 rotates in a certain direction of rotation for unwinding or winding the sensor arm 5 out of or into the winding groove 15, the endless spiral 53 rotates in such a way that the threading-in or threading-out point 22 between the two spiral windings, at which the sensor arm 5 is guided out of the winding groove 15 or the spiral inner volume, does not change with respect to the holder 42. The sensor arm 5 is always inserted into or removed from the winding groove 15 only at the insertion or removal point 22 which is predetermined by the endless spiral 52 and is fixed relative to the holder. Thus, the sensor arm 5 can be unwound from the winding groove 15 or rewound into it without interference.

FIG. 16 is a top view of the sensor arm 5 and the winding spiral 52 of the device 50 for on-site analysis from FIG. 15.

FIG. 17 is a perspective exploded view of yet another example of embodiment of a device 60 for on-site analysis according to the invention, and FIG. 18 is a perspective view of the device 60 for on-site analysis of FIG. 17 in an assembled state.

In the example shown, the spacing means has the form of a sheath 61 which at least partially envelops the sensor arm 5 and receives it in a longitudinally displaceable manner in a holder 62. Here, an outer side of the sheathing 61 is supported and preferably held on the holder 62, as can be clearly seen in FIG. 18.

In the present case, the sheathing 61 has a Teflon material on an inner side facing the sensor arm 5 or is formed from a Teflon material as a whole, but is not necessarily limited thereto. Unlike the sheath 8 of the previously described device examples 1, 30, 40 and 50, the sheath 61 envelops the sensor arm 5 within the entire holder 62 and spaces it therefrom.

FIG. 19 illustrates another perspective view of the sensor arm 5 including a schematically illustrated actuator 63 of the on-site analysis device 60 of FIG. 17, but without the holder 62.

In the present case, the actuator 63 has two drive rollers 64 which cooperate with the sensor arm 5 in a frictionally engaged manner in order to displace the latter in the longitudinal direction 7 between its extended measuring position and its retracted mounting position.

An example of embodiment of a device for on-site analysis according to the invention, which is not shown in the Figures but is similar to the device 60, has a linear (e.g. thread-like) traction means instead of the actuator 63 shown in FIG. 19, in particular instead of the drive rollers 64. The traction means is attached to a section of the sensor arm 5 enclosed by a sheath, in particular similar to the sheath 61. The sheathing has two open ends, from each of which an end portion of the traction means projects. In this case, an actuator interacts with the traction means in such a way that, for longitudinal displacement 7 of the sensor arm 5 into the measuring position, it (only) exerts a pulling force on an end section of the pulling means protruding from the first open end of the sheath and, for longitudinal displacement of the sensor arm into the mounting position, it (only) exerts a pulling force on an end section of the traction means protruding from the second end of the sheath. Thus, for each displacing movement of the sensor arm 5, the actuator always exerts a tensile force only on one of the two end sections of the traction means projecting from the sheathing. The traction means may be formed as a thread, that is, as a flexurally slack structure having a dominant one-dimensional extension and a uniformity in the longitudinal direction. The traction means can be formed from one fiber (e.g. nylon thread) or from several fibers (textile, synthetic fibers and the like).

FIG. 20 is a top view of an example of embodiment of a toilet device 110 according to the invention, and FIG. 21 is a perspective view of the toilet device 110 of FIG. 20.

The toilet device 110 is used for analyzing excrement, in particular human excrement, and, like the toilet device 100 shown in FIG. 1, has a collecting device 101, in this case in the form of a toilet bowl, for collecting the excrement. A toilet seat 120 pivotally attachable to attachment means 111 is shown in FIG. 22, which shows a perspective view of an example of embodiment of the toilet seat 120 according to the invention.

A device, such as one of devices 1, 30, 40, 50, or 60, for on-site analysis of excrement collected in the toilet bowl 101 of the toilet device 110 of FIGS. 20 and 21 is received in the toilet seat 120. Accordingly, the toilet seat 120 constitutes a housing of the device 1, 30, 40, 50 or 60. It is understood that the toilet seat 120 is pivotally attached to the toilet bowl 101 of the toilet device 110 of FIGS. 20 and 21 via attachment means 111.

Furthermore, the toilet seat 120 of the illustrated example of embodiment, and consequently the toilet device 110 of FIG. 20, also comprises a measuring device 112 received in the toilet seat 120 for measuring the geometry of a surface of the collection device 101. The measuring device 112 has at least one light source (e.g., LED, laser, etc.) not shown for generating light, at least one projection device 113 for projecting the light in a predetermined geometric pattern 114 (FIG. 20) onto the surface of the collection device 101, at least one camera 115 (optionally a further camera 115′ and/or 115″) for recording the geometric pattern 116 imaged on the surface of the collection device 101, and an electronic control device (not shown). The control means is adapted to determine the surface geometry or topology of the collection device 101 from a deviation (distortion) of the captured imaged geometric pattern 116 from the originally projected geometric pattern 114.

The toilet seat 120 may also accommodate other sensors, for example (capacitive) contact sensors 117, ultrasonic sensors 118, temperature sensors, pressure sensors and the like, in addition to the measurement device 112 and/or the on-site analysis device 1, 30, 40, 50, 60. It is to be understood that the type, number and arrangement of the sensors on the seat 120 may differ from the ones shown in FIG. 22, so that FIG. 22 is not a limitation in this respect, but is to be regarded merely as a possible exemplary combination and configuration.

FIG. 23 illustrates two different perspective views A and B of an example of embodiment of a self-sealing cartridge 130 for sample carriers, e.g., the sample carriers 6, according to the invention; FIG. 24 illustrates a perspective exploded view of the cartridge 130 of FIG. 23.

The self-sealing cartridge 130 is used to provide sample carriers, for example sample carriers 6, for an apparatus for on-site analysis of excrements, for example apparatuses 1, 30, 40, 50, 60. The cartridge 130 comprises a cartridge housing 131 accommodating the sample carriers 6. The cartridge housing 131 is formed of a first housing half 132 and a second housing half 133. The first housing half 132 and the second housing half 133 are held so as to be displaceable relative to one another between a closed position and a dispensing position. In the closed position, the sample carriers 6 accommodated in the cartridge housing 131 are shielded, preferably hermetically shielded, from an external environment. In the dispensing position, one of the sample carriers 6 accommodated in the cartridge housing 131 can be removed from the housing 131. By means of a spring element 134, e.g. a helical spring, the two housing halves 132, 133 are automatically biased into the closed position.

In FIG. 24, it can be seen that in the present example of embodiment, the spring element 134 is mounted at one end on the first housing half 132 and presses at the other end on a feed plunger 135 displaceably mounted in the second housing half 133, which presses the sample carriers 6 or a stack thereof received in the cartridge housing 131 against an upper housing wall of the second housing half 133. In the region of the sample carrier 6 resting against the upper housing wall of the second housing half 133, a dispensing window 136 is provided in the second housing half 133 for dispensing the sample carrier 6 resting against the upper housing wall from the cartridge housing 131. In the closed position of the cartridge housing 131, the dispensing window 136 is closed by a wall portion 137 of the first housing half 132. In the dispensing position, the dispensing window 136 is uncovered to removably provide the sample carrier 6 resting against the upper housing wall of the second housing half 133 from the cartridge housing 131 through the dispensing window 136.

Furthermore, in the example of embodiment shown in FIGS. 23, 24, an entrance window 138 is provided in the first housing half 132 in the region of the sample carrier 6 resting against the upper housing wall of the second housing half 133, which entrance window 138 is closed in the closed position by a wall section 139 (on the rear side in FIG. 24) of the second housing half 133. In the dispensing position, the entrance window 138 is uncovered. Furthermore, it is arranged in such a way that by inserting an ejection plunger 140, which is merely symbolized in FIG. 24, through the entrance window 138, the sample carrier 6, which rests against the upper housing wall of the second housing half 133, can be dispensed from the cartridge housing 131 through the dispensing window 136.

Furthermore, the cartridge 130 has an electronic memory chip 141 on which data relevant to the sample carrier, such as indication, production date, expiration date, batch number, number of sample carriers, etc., are stored. The memory chip 141 may be writeable for updating the number of remaining sample carriers 6 after each sample carrier removal. The memory chip 141 may, for example, be designed as an RFID chip.

FIG. 25 illustrates a perspective view of an example of a sample carrier dispensing device 150 for receiving the cartridge 130 of FIG. 23. The sample carrier dispensing device 150 has a cartridge receptacle 151 for interchangeably receiving the cartridge 130.

FIG. 26 shows in a perspective view A the cartridge 130 of FIG. 23 received in the sample carrier dispensing device 150 of FIG. 25 in the dispensing position of the cartridge 130, and in a perspective view B the sample carrier dispensing device 150 of view A in the closed position of the cartridge 130.

As can be seen in view A, the ejection plunger 140 pushes the two housing halves 132, 133 apart through the entrance window such that the wall portion 139 of the second housing half 133 and the wall portion 137 of the first housing half 132 respectively expose the entrance window 138 and the dispensing window 136. The sample carrier 6 resting against the upper housing wall of the second housing half 133 is then dispensed from the dispensing window 136 by the ejection plunger 140. Subsequently, the ejection plunger 140 is completely retracted from the entrance window 138, so that the cartridge 130 automatically closes again as a result of the elastic force of the spring element 134 acting on the two housing halves 132, 133; that is, the two wall sections 137, 139 completely close the dispensing window 136 and the entrance window 138, respectively. This state of the closed position is shown in view B of FIG. 26.

At this point, it should be explicitly pointed out that features of the solutions described above or in the claims and/or figures can also be combined, if desired, in order to be able to implement or achieve the explained features, effects and advantages in a correspondingly cumulative manner.

It is understood that the examples of embodiment explained above are merely several possible embodiments of the devices according to the invention. Consequently, the invention is not limited to these examples of embodiment. Equally effective further embodiments result from technically useful further combinations of the features described herein. In particular, the features and combinations of features mentioned hereinabove in the general specification and the description of the figures and/or shown alone in the figures are usable not only in the respective combinations explicitly indicated herein, but also in other combinations or on their own, without departing from the scope of the present invention.

All features disclosed in the application documents are claimed to be essential to the invention insofar as they are new, individually or in combination, over the prior art.

### LIST OF REFERENCE NUMBERS

1 on-site analysis device
2 housing
3 holding means
4 measuring location
5 sensor arm
5′ sensor arm windings
6 sample carrier
7 longitudinal direction
8 sheathing
9 bending portion
10 holder
11 winding roller
12 circumferential direction
13 driving shaft
14 radial direction
15 winding groove
16 limiting web
17 retaining claw
18 upper groove wall
18′ lower groove wall
19 groove width
20 axial direction
21 engagement element
22 threading-in/threading-out point
30 on-site analysis device
31 winding roller
40 on-site analysis device
41 winding roller
42 holder
43 sinous spring
50 on-site analysis device
51 winding roller
52 endless spiral
53 direction of rotation
60 on-site analysis device
61 sheathing
62 holder
63 actuator
64 drive roller
100 toilet device
101 collecting device, toilet bowl
102 bowl rim
103 toilet seat
110 toilet device
111 fastening device
112 measuring device
113 projection device
114 projected geometric pattern
115 camera
115′ optional camera

1. Device for on-site analysis of excrements with a sensor device by means of which an excrement sample can be detected at a measuring location and at least partially analyzed, the sensor device having a bendable, preferably elastically bendable, sensor arm which can be mounted on a holder at least partially bent, in particular for a sample carrier displaceable in the longitudinal direction by means of an actuator between an extended measuring position bridging a distance between the holder and the measuring location for detecting the excrement sample and a retracted mounting position mounted at least partially bent on the holder; characterized in that the sensor arm is spaced by at least one spacing means from such portions of the holder in relation to which the sensor arm moves in the direction of its longitudinal displacement.

2. Device according to claim 1, characterized in that the sensor arm is wire-shaped.

3. Device according to claim 1, characterized in that a winding roller is rotatably mounted in the circumferential direction on the holder, the winding roller having a circumferentially extending winding groove which is recessed in the radial direction of the winding roller and into which the sensor arm can be inserted and removed, the spacing means being set up and arranged to selectively enable or prevent the insertion and/or removal of the sensor arm into or from the winding groove, respectively.

4. Device according to claim 3, characterized in that the spacing means and/or at least one groove wall limiting the winding groove is/are elastically deformable such that by selectively applying a predetermined deformation force on the spacing means and/or on the groove wall, the winding groove is uncovered and otherwise blocked.

5. Device according to claim 4, characterized in that the predetermined deformation force can be applied solely by means of the sensor arm by applying a predefined pulling and/or pushing force to it.

6. Device according to claim 4, characterized in that the holder has an engagement element that is operably engageable with the spacing means and/or with the at least one groove wall and that exerts the predetermined deformation force on the spacing means and/or on the groove wall.

7. Device according to claim 3, characterized in that the spacing means is at least one retaining claw fastened to the one groove wall and extending, with a free end, in the direction of a further groove wall opposite to the one groove wall.

8. Device according to claim 7, characterized in that in the circumferential direction of the winding groove, several retaining claws are arranged, the retaining claws being fastened at one end in alternating sequence to the one groove wall and to the further groove wall opposite the one groove wall.

9. Device according to claim 3, characterized in that the spacing means is a sinous spring or endless spiral arranged in the winding groove and circumferentially surrounding the winding groove.

10. Device according to claim 9, characterized in that the sinous spring, in its circumferential direction of extension, is arranged so as to be slidable in the longitudinal direction in relation to the winding groove or in that the endless spiral is arranged rotatably about its longitudinal axis.

11. Device according to claim 3, characterized in that a nominal groove width of the winding groove is defined such that successive windings of the sensor arm in the winding groove can be arranged in the winding groove adjacent to one another only in the radial direction.

12. Device according to claim 1, characterized in that the one or an additional spacing means is a sheathing which envelops the sensor arm at least in portions and receives it so that it can slide in the longitudinal direction, an exterior of the sheathing 8 being supported, preferably held, at the holder.

13. Device according to claim 3, characterized in that the actuator rotationally drives the winding roller so as to displace the sensor arm between its mounting position and its measuring position.

14. Device according to claim 1, characterized in that the actuator has at least one drive roller which cooperates with the sensor arm in a frictionally engaged manner in order to displace the latter between its extended measuring position and its retracted mounting position.

15. Device according to claim 11, characterized in that a linear pulling means is fastened to a section of the sensor arm enveloped by the sheathing, which protrudes from a first open end of the sheath and an opposite second open end of the sheath, the actuator cooperating with the pulling means in such a way that, for longitudinal displacement of the sensor arm into the measuring position, it exerts a traction force on a section of the pulling means protruding from the first open end of the sheath, and for longitudinal displacement of the sensor arm into the mounting position, it exerts a pulling force on a section of the pulling means protruding from the second end of the sheath.

16. Device according to claim 1, characterized in that it is accommodated in a housing which is a toilet seat that can be fastened to a toilet bowl for receiving the excrements.

17. Device according to claim 1, characterized in that it is accommodated in a housing which can be fastened via a retention means to a toilet bowl for receiving the excrements.

18. Device according to claim 1, characterized by a self-sealing cartridge for providing several sample carriers received in a cartridge housing formed by a first housing half and a second housing half, the first housing half and the second housing half being held so as to be displaceable relative to one another between a closed position, in which the sample carriers accommodated in the cartridge housing are shielded, in particular hermetically shielded, from an external environment, and a dispensing position, in which at least one of the sample carriers accommodated in the cartridge housing can be removed from the housing; and being biased into the closed position by a spring element.

19. Device according to claim 18, characterized in that the spring element is mounted at one end on the first housing half and presses at the other end on a feed plunger displaceably mounted in the second housing half, which presses the sample carriers received in the cartridge housing against a housing wall of the second housing half; in the region of the sample carrier resting against the housing wall, a dispensing window being provided in the second housing half for dispensing the sample carrier resting against the housing wall from the cartridge housing; in the closed position, the dispensing window being closed by a wall portion of the first housing half and in the dispensing position, the dispensing window being uncovered to removably provide the sample carrier resting against the housing wall of the second housing half from the cartridge housing through the dispensing window.

20. Device according to claim 19, characterized in that an entrance window is provided in the first housing half in the region of the sample carrier resting against the housing wall of the second housing half, which entrance window is closed in the closed position by a wall section of the second housing half and is uncovered in the dispensing position and arranged in such a way that by inserting an ejection plunger through the entrance window, the sample carrier, which rests against the housing wall of the second housing half, can be dispensed from the cartridge housing through the dispensing window.

21. Self-sealing cartridge for providing sample carriers for a device for the on-site analysis of excrements, having a cartridge housing receiving the sample carriers and formed by a first housing half and a second housing half, the first housing half and the second housing half being held so as to be displaceable in relation to one another between a closed position, in which the sample carriers accommodated in the cartridge housing are shielded, in particular hermetically shielded, from an external environment, and a dispensing position, in which one of the sample carriers accommodated in the cartridge housing can be removed from the housing; and being biased into the closed position by a spring element.

22. Cartridge according to claim 21, characterized in that the spring element is mounted at one end on the first housing half and presses at the other end on a feed plunger displaceably mounted in the second housing half, which presses the sample carriers received in the cartridge housing against a housing wall of the second housing half; in the region of the sample carrier resting against the housing wall, a dispensing window being provided in the second housing half for dispensing the sample carrier resting against the housing wall from the cartridge housing; in the closed position, the dispensing window being closed by a wall portion of the first housing half and in the dispensing position, the dispensing window being uncovered to removably provide the sample carrier resting against the housing wall of the second housing half from the cartridge housing through the dispensing window.

23. Cartridge according to claim 22, characterized in that an entrance window is provided in the first housing half in the region of the sample carrier resting against the housing wall of the second housing half, which entrance window is closed in the closed position by a wall section of the second housing half and is uncovered in the dispensing position and arranged in such a way that by inserting an ejection plunger through the entrance window, the sample carrier, which rests against the housing wall of the second housing half, can be dispensed from the cartridge housing through the dispensing window.

24. Toilet device for analyzing excrements, having a collecting device for collecting the excrements, characterized in that a device for on-site analysis of the excrements according to claim 1 is fastened to the collecting device.

25. Toilet device according to claim 24, characterized in that a measuring device for measuring the geometry of a surface of the collecting device is provided which has at least one light source for producing light, at least one projecting device for projecting the light onto the surface of the collecting device in a predetermined geometric pattern, at least one camera for recording the geometric pattern imaged on the surface of the collecting device and an electronic control device adapted to determine the surface geometry of the collecting device from a deviation of the recorded imaged geometric pattern from the originally projected geometric pattern.

26. Toilet device according to claim 25, characterized in that two cameras are provided and arranged to record the geometric pattern imaged on the surface of the collecting device from various imaging directions.

27. Toilet device according to claim 25, characterized in that two projecting devices are provided and arranged to project the predetermined geometric pattern onto the surface of the collecting device from different projection angles.

28. Toilet device according to claim 24, characterized in that the collecting device is a toilet bowl with a toilet seat attached to it, the device being accommodated in the toilet seat for on-site analysis of the excrements.

29. Toilet device according to claim 24, characterized in that the collecting device is a toilet bowl with a toilet seat attached thereto, the measuring device for measuring the geometry of the surface of the collecting device being accommodated in the toilet seat.

30. Toilet seat for a toilet device with a toilet bowl for collecting excrements, characterized in that in the toilet seat, a device for on-site analysis of the excrements collected in the toilet bowl according to claim 1 is accommodated.
